(19) United States
(12) Patent Application Publication
(10) Pub. No.: US 2018/0214400 A1
(43) Pub. Date: Aug. 2, 2018

(54) THE USE OF KAURANES COMPOUNDS IN THE MANUFACTURE OF MEDICAMENT FOR TREATMENT OF CADIAC HYPERTROPY AND PULMONARY HYPERTENSION

(71) Applicants: Wen TAN, Dongguan (CN); KEY-PHARMA BIOMEDICAL INC., Dongguan (CN)

(72) Inventor: WEN TAN, Dongguan (CN)

(21) Appl. No.: 15/932,505

(22) PCT Filed: Sep. 7, 2016
(86) PCT No.: PCT/CN2016/098304

§ 371 Date: Mar. 7, 2018

Related U.S. Application Data
(60) Provisional application No. 62/216,658, filed on Sep. 10, 2015.

(51) Int. Cl. A61K 31/19; A61P 9/00; A61P 15/10; A61K 9/00; A61K 9/08

(52) U.S. Cl. CPC A61K 31/19; A61P 9/00; A61K 9/08; A61K 9/0019; A61P 15/10

(57) ABSTRACT

The invention relates novel pharmaceutical use of kaurane compounds of formula (I) in treating and preventing cardiac hypertrophy and myocardium remodeling. The said compounds also can significantly ameliorate pulmonary hypertension and preventing vascular hypertrophy. The said compounds can also be used to suppress fibrosis and to treat erection dysfunction, neurological degenerations and other related diseases by modulating cGMP or cAMP signal pathway and/or by reducing reactive oxygen species (ROS). Wherein R¹: hydrogen, hydroxyl or alkoxy. R²: carboxyl, carboxylate, acyl halides, aldehyde, methyl-hydroxyl, and ester, acylamide, acyl or ether group hydrolysable to carboxyl. R³, R⁴, R⁵, R⁶, R⁸: independently, oxygen, hydroxyl, methyl-hydroxyl, and ester or alkoxymethyl group hydrolysable to methyl-hydroxyl. R⁷: methyl, hydroxyl, and ester or alkoxymethyl hydrolysable to methyl-hydroxyl. R⁹: methylene or oxygen.

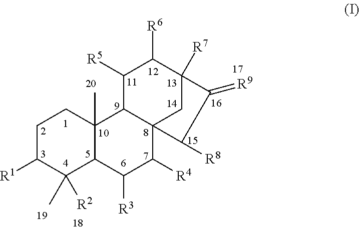

### BACKGROUND

Cardiac hypertrophy is a compensatory response to pressure-overload (Hilfiker-Klemer et al, JACC. 2006; 48(9):A56-A66.). It will eventually enter into a decompensate state with deterioration of cardiac function. Under the stimulation of increased pressure, this transition process from compensate to decompensate state often involves in cardiac remodeling (Konstam et at., JACC Cardiovascular imaging. 2011; 4(1):98-108). Cardiac remodeling is a complex process involving cardiac myocytes overgrowth or death, vascular rarefaction, fibrosis, inflammation, and progressive cardiac dysfunction (Burchfield et al. Circulation. 2013; 128(4):388-400). Increment in extracellular matrix and associated collagen network surrounds each cardiac myocyte raise cardiac stiffness. Disturbance of the interstitial network and fibrosis impairs contractile function and contributes to adverse myocardial remodeling after hypertensive heart disease, Cardiac fibroblasts, the most abundant cell type in the heart (constituting two-thirds of the total cell population), are responsible for extra cellular matrix (ECM) deposition and create the scaffold for cardiomyocytes. Activated myofibroblasts result in over-production of ECM, predominantly collagen types I and III, into the interstitial and perivascular space. Excessive collagen deposition leads to myocardial stiffening, impaired cardiac re-laxation and filling (diastolic dysfunction), and overload of the heart.

Studies showed that increased interstitial collagen and cardiac fibrosis may not the only detriments contribute to cardiac dysfunction in hypertrophy. Other mechanisms such as neuro-hormonal activation, electrophysiological remodeling and autonomic imbalance with increase in sympathetic activity and withdrawal of vagal activity may also contribute to the deteriorated cardiac function. Preventing pathological cardiac hypertrophy and cardiac remodeling is an important therapeutic goal to preserve the cardiac function from deterioration.

It has been reported that increase of cGMP by blocking PDE-5 with sildenafil suppresses both chamber and cardiomyocytes hypertrophy, and improves in vivo heart function in mice exposed to chronic transverse aortic constriction (Yuan F. JMCC. 1997; 29(10):2837-48). Sildenafil also reversed pre-established hypertrophy induced by pressure load while restoring chamber function.

In addition, the deterioration of left heart in TAC rats will in turn, causes hypoxia and increased pressure within pulmonary arteries and cause vascular remodeling (Chen et al., Hypertension. 2012; 59:1170-1178.).

The narrowing of pulmonary arteriole will results an increase in resistance and lead to pulmonary hypertension. Pulmonary hypertension (PH) is a rapidly progressive disease of the pulmonary vasculature, which subsequently leads to right heart failure. PH is provoked by prolonged exposure to hypoxia, which leads to structural remodeling of pulmonary vessels. The combination of vasoconstriction and vascular remodeling, results in PHT plexogenic pulmonary arteriopathy which is characterized by medial hypertrophy, intimal proliferation, and fibrosis of small muscular arteries, synthesis and deposition of collagen, muscularization of pre-capillary vessels as well as the diagnostic plexiform lesion. The lung is an organ with abundant PDE-5 expression (Burchfield et al Circulation. 2013; 128(4):388-400). It has been shown that sildenafil, PDE-5 inhibitor, attenuated the rise in pulmonary artery pressure and vascular remodeling when it was given before chronic exposure to hypoxia and during ongoing hypoxia-induced PHT in rats (Kwong et al., Cell metabolism. 2015; 21(2):206-14). Clinical investigations in patients with PHT also indicated that sildenafil therapy helps improve patient's condition.

PDE-5 is an enzyme that catalyzes the hydrolytic degradation of cyclic GMP—an essential intracellular second messenger that modulates diverse biological processes in living cells. Three selective inhibitors of PDE-5—sildenafil, vardenafil and tadalafil—have been successfully used by millions of men worldwide for the treatment of erectile dysfunction. As noted above, sildenafil and tadalafil are currently used for the treatment of cardiac hypertrophy, cardiomyopathy, pulmonary hypertension, other circulatory disorders. Recent studies suggest potential neurological applications of PDE-5 inhibitors, including, cardiac hypertrophy, cardiomyopathy, stroke, neurodegenerative diseases.

PDE-5 inhibitors may also protect the brain against stroke and other neurodegenerative diseases. Oral treatment with sildenafil for seven consecutive days starting 2 h or 24 h after embolic middle cerebral artery occlusion significantly enhanced neurological recovery without any effect on infarct volume. The authors proposed that an increase in the cortical levels of cGMP after sildenafil treatment may have evoked neurogenesis and reduced neurological deficits.

However, sildenafil may possess sever adverse effects for patients. There is unmet medical need for new generation of PED for prevention fibrosis in cardiac and lung tissue with high efficient and low toxicity.

Compound A is a beyerane diterpene derived from stevioside which is known for its sweet taste and effects on the cardiovascular system in traditional medicines in South America (Geuns J M C. Stevioside. Phytochemistry. 2003; 64(5):913-21). In Prior art, studies reveal that the kauran like compound such as compound A and compound B possesses cardioprotective effect in acute ischemia-reperfusion heart injuries and reduces arrhythmia (Tan, U.S. patent Ser. No. 11/596,514, 2006). It is also reported that isosteviol (compound A) may be beneficial to diabetes. However, the effects of kuarane compounds such as compound A on cardiac or vascular remodeling, or on cardiac hypertrophy and pulmonary hypertension which is characterized by vascular hypertrophy, vessel muscularization and collagen deposition has never been reported. The effects of compounds of formula (I) and isosteviol (compound A) on cGMP or TGF-β which are known factors involved in cardiac hypertrophy or fibrosis have been reported in prior arts.

In this invention we presented for the first time that Kaurane like compounds of formula (I), such as compound A, are useful for treatment of cardiac hypertrophy in TAC-induced hypertrophy rats. It can also prevent cardiac remodeling by reducing the fibrosis and collagen deposition, and the size of cardiomyocytes. In addition, Kaurane like compounds such as compound A can also prevent pulmonary hypertrophy in the same TAC-induced hypertrophy rats. The role of Kaurane like compounds such as compound A involves both enhanced cGMP signal pathway and scavenging of ROS. Furthermore, the invention disclosed a superior therapeutically effects of compound A over other drugs and the compound A involve other phosphodiesterases or mechanisms.

### DETAIL OF INVENTION

The invention discloses the effects of kaurane compounds as in formula (I) in treating cardiac hypertrophy and pulmonary hypertension. The compounds in formula (I) represent a class of natural, synthetic or semi-synthetic compounds. Many of these compounds has been known to public (Kinghorn A D, 2002, p 86-137; Sinder B B, et al., 1998; Chang F R et al., 1998; Hsu, F L et al., 2002). Compounds in formula (I) may have one or more asymmetric centers and may exist in different stereoisomers.

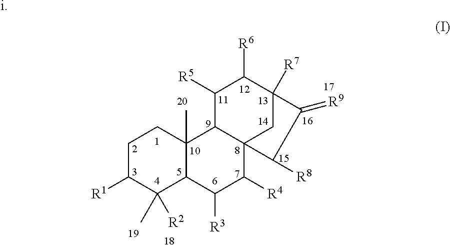

Wherein
ii. R¹: hydrogen, hydroxyl or alkoxy
iii. R²: carboxyl, carboxylate, acyl halide, aldehyde, methyl-hydroxyl, and ester, acylamide, acyl or ether group hydrolysable to carboxyl.
iv. R³, R⁴, R⁵, R⁶, R⁸: independently, oxygen, hydroxyl, methyl-hydroxyl, and ester or alkoxymethyl hydrolysable to methyl-hydroxyl.,
v. R⁷: methyl, hydroxyl, and ester or alkoxymethyl hydrolysable to methyl-hydroxyl.
vi. R⁹: methylene or oxygen.

A group of preferred compounds is presented in Formula (I′). The said compounds have kaurane structure, with substitutions adjacent to carbon 13, and derivatives at carbons 17 and 18. These said compounds may have multiple asymmetric centers, and exist as different stereo-isomers or dia-stereo-isomers. The absolute configuration related the position 8 and 13 are (8R,13S) or (8S,13R).

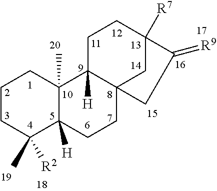

Wherein:
vii. R²: carboxyl, carboxylate, aldehyde, methyl-hydroxyl, methyl ester, acyl methyl, acyl halides.
viii. R⁷: methyl, methyl-hydroxyl, or methyl ether.
ix. R⁹: methylene or oxygen.

Compound A can be obtained by acidic hydrolysis of natural stevioside. Compound B is the aglycone of stevioside which is compound B glycoside. Compound A and B are isomers. Compound B can be obtained from stevioside by chemical reactions of hydrolysis and oxidation or by catanalysis reactions of bacteria within animal intestine.

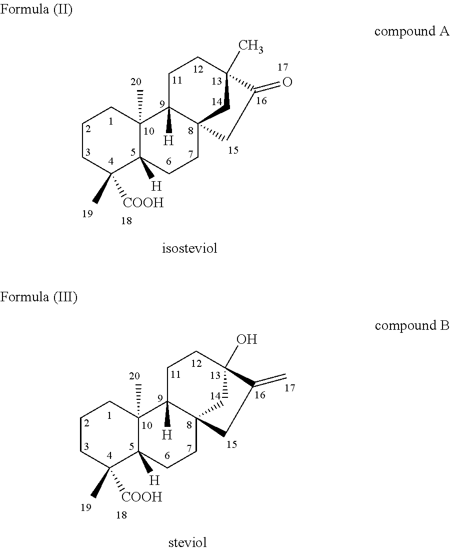

Compound A, molecular formula, C₂₀H₃₀O₃; chemical name: (4α,8β,13β)-13-methyl-16-oxo-17-norkauran-18-oic acid; It also named compound A, ent-16-ketobeyran-18-oic acid. The said compound is a tetracyclic diterpene with kaurane structure, wherein, the absolute configuration of asymmetric carbons are: (4R,5S,8R,9R,10S,13S), a substituted methyl group at carbon 13, a carbonic group at carbon 16 and a carboxyl group at carbon 18(Rodrigues et al., 1988).

Compound B, molecular formula, C₂₀H₃₀O₃ chemical name: ent-13-hydroxykaur-16-en-18-oic acid, it also named as steviol, the said compound is also a tetracyclic diterpene with kaurane skeleton, wherein, the absolute configuration of chiral carbons are: (4R,5S,8R,9R,10S,13S), a substituted hydroxyl group at carbon 13, a methylene group attached by a double bond adjacent to carbon 16 and carboxyl group at carbon 18(Rodrigues et al., 1993).

Compound A or B may also exist as carboxylate at 18 position, wherein the carboxylate are sodium and basic metals or chloride and halogen. Both compound A and B have the kaurane structure and are kaurane compounds. Compound A is the more preferred compound in this invention. This invention discloses that compound A or B has similar therapeutic effects in treating and preventing cardiac hypertrophy ad pulmonary hypertension. It may be inferred that all the other compounds of formula (I) also have the same kind of therapeutic effects as did of compound A. It is reported that large amount of compound B may be mutagenic under certain condition in vitro, therefore, compound A is more preferable comparing with compound B, to be used in pharmaceutical medication.

Compound A used in this invention is a sodium salt of compound A with a better solubility.

Kaurane compounds of formula (I) have been widely studied for their possible biological and pharmacological effects. Most of the studies in art concern their roles in metabolite mechanism (Kinghorn, A D. 2002, Stevia, by Taylor & Francis Inc.).

For instance, it was reported that the said compounds affects cellular metabolite, glucose absorption in intestine and carbohydrate metabolism, energy metabolism in mitochondria of hepatic cells, and metabolite of carbohydrate and oxygen in renal cells. It was also reported that the said compounds cause vasodilation and hypotension. More recently it was revealed the effects of compound A on cardiac and cerebral ischemia, arrhythmia, cardiac contractility in ischemia heart. No study in art has documented the effects of Kaurane compounds of formula (I) or compound A on cardiac hypertrophy, fibrosis and pulmonary hypertension. Furthermore, no prior arts disclosed that Kaurane compounds of formula (I) act as phosphodiesterase inhibitors or ROS scavengers.

This invention disclosed that TAC induced cardiac hypertrophy and myocardial remodeling rats. 1) Compound A could significantly inhibit myocardial hypertrophy after 3 weeks of TAC; 2) Compound A could significantly improve cardiac functions without increased in cytosolic Ca²⁺, improve electrophysiological remodeling; 3) Compound A could inhibit cardiac fibrosis in vivo and TGF-β₁-induced fibroblast proliferation in vitro; 4) Compound A can prevent pulmonary hypertension as result of TAC as indicated by significantly inhibiting media hypertrophy of lung vessel and production of collagen; 5) Compound A can significantly reduce the increased size of myocardium induced by isoproterenol; 6) Compound A acted through the elevation of cGMP by inhibition of PED; 7) The cardioprotective effects of compound A were superior than the PDE-5A Inhibitor sildenafil, which indicating an additional novel mechanism is involved. 8) Compound A was found also modulating both cAMP and cGMP in either 2′3′ciclic or 3′5′ciclic formation in fibroblasts or cardiomyocytes.

This invention disclosed that compound A reduced the effects of TAC-induced cardiac hypertrophy and cardiomyocyte dilation as well as the proliferation of myofibroblasts. A significant increase in heart to body weight ratio (HW/BW), an index of cardiac hypertrophy, was observed in the 3-week TAC group. The increase in HW/BW was greatly reduced in TAC with compound A treatment. The increased HW/BW was accompanied by increased cardiomyocyte cross-sectional area which was increased for 76% percent in 3 week TAC rats comparing to Sham rats. It was increased only for 10% in 3 weeks TAC rats treated with compound A, along with a significant improved cardiac function either systolic or diastolic. The cardiac and cardiomyocyte hypertrophy was ameliorated by compound A.

Concurrent with hypertrophy changes were the formation of collagen and actin remodeling. A well-characterized histological structure change in TAC rats is its actin cytoskeleton dynamics, i.e. a higher F-to-G actin content ratio. TAC induced polarization of actin that increases the ratio of polymer (F-actin) to monomer (G-actin). Pressure overload on the ventricles also triggers interstitial fibrosis, increased cardiac collagen deposition.

This invention disclosed that compound A treatment reduced F-actin level and the deposition of collagen. In addition, this invention disclosed that compound A is more effective and potent than sildenafil in effects noted above.

The reduction of fibrosis and collagen deposition led to an increase in myocardial compliance and contractility which results a better performance of heart as blood pump as measured by higher elasticity and lower stiffness of left ventricular during contraction and dilation.

The left ventricular pressure and volume were measured simultaneously. Tow parameters can be derived by studying of the relationship of pressure-volume during changes of either preload or afterload. ESPVR, the slope of end-systolic pressure-volume relationships which represent end-systolic elastics; EDPVR, the slope of end-diastolic pressure-volume relationship which, represents cardiac stiffness. In hypertrophy hearts after 3 or 9 weeks TAC, the cardiac pump dysfunction was manifested by a significant decreased ESPVR and increased in EDPVR. This invention disclosed that treatment with compound A in TAC rats prevented the deteriorations in both of ESPVR and EDPVR as well as the systolic and diastolic function comparing to sham control rats. Therefore, compound A are useful to preserve a normal elasticity during contraction and reduce diastolic stiffness of hearts with high pressure load as in TAC rats.

It has been demonstrated that TGF-β signaling pathway plays a critical role in myocardial fibrosis following pressure overload, mediating collagen production. The cGMP signaling pathway plays a key regulatory role against TGF-β-induced cardiac fibrosis.

This invention disclosed that compound A can prevent TGF-β induced proliferation in cultured neonatal rat cardiac fibroblasts. Furthermore, this invention disclosed that there were a significant increase in cGMP levels in compound A treated cardiac fibroblasts which is related to its anti-hypertrophy and anti-fibrosis roles.

Furthermore, this invention disclosed that microRNA21, which has been demonstrated as a promoter of cardiac fibrosis, was significant reduced by compound A at the penumbra region of the ischemic heart. This changes is mircoRNA21 was along with a significant amelioration of fibrosis at the same region. This effect of compound A has never been reported in prior art.

BNP is an important marker for hypertrophy. Hypertrophic response of cardiomyocytes to isoproterenol stimulus was accompanied with increase in mRNA expression of BNP as demonstrated with reverse transcriptase polymerase chain reaction (RT-PCR), and BNP protein as demonstrated by western blot. This invention disclosed that treatment of compound A can greatly reduce the increase of both BNP production and BNP mRNA expression in cardiomyocytes.

The increase of cGMP could be the results of either stimulating of BNP or inhibition of phosphodiesterase (PDE). The enhancing effects of cGMP by compound A are mainly due to an inhibition of PDE since BNP were reduced by compound A.

There are both cAMP and cGMP and their isomers may play roles in intracellular signal pathway. Using HPLC-MS method one can detect cAMP and cGMP isomers produced by different cells at same time. This invention disclosed that there were significant changes in 3′5′cGMP, 2′3′cGMP 3′5′cAMP and 3′5′cAMP levels in hypertrophy cardiomyocytes, normal cardiomyocytes and fibroblasts after compound A treatments. The changes were different with different time of incubations with compound A. These indicated that the different cAMP or cGMP and isomers are involved in the effects of compound A in treatment of fibrosis, hypertrophy and other decreases. These effects of compound A have never been reported in prior arts.

This invention also disclosed the use of compound A in treatment of pulmonary hypertension. Pressure overload induced by TAC is one of the established methods to induce pulmonary hypertension in rats. This invention demonstrated pulmonary hypertensive damages in the same TAC animals mention above. Considerable lung vascular remodeling was evident in pulmonary hypertension rats in medial wall thickening in either in small (inner diameter <100 um) or medium pulmonary arteries (diameter <100 um). This invention disclosed that compound A treatment prevented vascular remodeling in both small and medium arteries. The degree of muscularization were categorized into non-muscularization, partially muscularization and fully muscularization. After treatment of compound A, the number of non-muscularization vessels were increased, which indicating an amelioration of pulmonary hypertension. Compound A is more effective than sildenafil in this regard.

This invention also disclosed the use of compound A in treatment of cardiac hypertrophy, fibrosis and cardiomyopathy and renal fibrosis in diabetes.

In addition, Mitochondrial-derived ROS may function as intracellular messengers to modulate cardiac hypertrophy signaling pathways. Daofu Dai reported that ROS directly produced in mitochondria can be the pivotal mediator of Gaq-induced cardiac hypertrophy (Dai D F, Rabinovitch P. Autophagy. 2011; 7:917-918).

In this invention, we disclosed that compound A could suppress cardiomyocytes hypertrophy by reducing ROS (reactive oxygen species) in either cytosol and mitochondria in addition to the inhibition of PED, while classic PED inhibitor such as sildenafil has no such effects been reported in prior arts. This explains the superiority of compound A over sildenafil in suppressing hypertrophy and other diseases. This invention disclosed a new use of compound A as PED inhibitor with novel mechanism which is different than what been disclosed in prior art.

This invention demonstrated that the compound A was more potent than sildenafil in suppressing cardiac hypertrophy and collagen deposition as well as in stimulation of cGMP production, while Sildenafil is the first line drug for erection dysfunction. In an embodiment, this invention reveals a long lasting penile erection in male rats and dogs after treatment with relative higher dose of compound A. This invention also disclosed that compound A can be used for erection dysfunction.

This invention also disclosed that compound A can be used for treatment of Alzheimer's disease. In prior arts, it was reported that enhancement of cGMP signal pathway by sildenafil (Rc Kukreja, et al. exp clin cardiol 2011; 16(4):e30-e35). Our invention showed that compound A is more potent than sildenafil in stimulating cGMP. This invention demonstrated anti-astrogliosis and anti-scar-forming effects of compound A in cerebral injured rats by compound A. This invention disclosed that compound A can be used to prevent neurodegenerative disease, dementia such as Alzheimer's disease.

In prior art, it was disclosed that the therapeutic effects of compounds A or B above mention may involve in multiple mechanisms. Wang K L. suggested that hypotensive effects of compound A may involve potassium channels of smooth muscle cell membrane (Wang, K L et al, 2004), while Jeppesen P B. demonstrated that potassium channels potassium channels were not involved in a stimulating effects of compound A on insulin secretion (Jeppesen P B., et al, 2000). Tan disclosed that compound A and B play protective roles in ischemic mitochondria, which can only be partially blocked by 5-OH-decdanoate, a potassium ATP channel blocker (Tan, U.S. patent Ser. No. 11/596,514, 2006). Therefore, in prior art it is not clear whether and how compound A is related with KATP channels.

This invention disclosed exclusively that compound A per se had no effect on either sarcolemma or mitochondrial KATP channel. Instead, compound A is acting only as a sensitizer which render the KATP channel response greater to known KATP channel openers, such as pinacidil and to change of ATP.

In prior art, it disclosed that compound A can enhance the contractility and protect the ischemic cardiomyocytes. However, all the known inotropic medicine enhance the cardiac function on the expanses of increase Ca++, which in turn increase the consumption of oxygen. Therefore the use of inotropic medicine would worsen the cardiac condition as indicated by depressed or elevation of ST wave from baseline in ECG. In prior art, only the inotropic effects of compound A were disclosed.

This invention disclosed a novel use of inotropic medicine selectively that is compound A can be used to improve the cardiac function in a deteriorative hypertrophy heart without increase cytosol Ca++ or oxygen consumption. In addition it was not worsening the ECG instead it improve the ECG in hypertrophy heart. This is due to that compound A can reduce cardiomyocytes cytosol Ca++ level but enhance only the peak of Ca++ transient during each contraction in hypertrophy cardiomyocytes. This novel finding makes compound A different from other known traditional inotropic medicine such digitalis and beta agonists such as epinephrine.

This invention also disclosed that in cardiomyocyte from guinea pig, that compound A can reduce elongated QT segment and increased QT variations, further it prevent prolonged action potential, decrease resting potential and suppressed Herg (Ikr) currents as result of ischemia and reperfusion. Compound A can also as an scavenger to reduce ROS (reactive oxygen species). Therefore, it can be used for treatment of abnormal ECG in clinic diagnosed with above or used for diseases or clinic procedures which may involve above mentioned mechanism.

In other embodiment, the invention disclosed compound A is effective against late phase or long term cerebral damage by inhibition of astrogliosis. In prior art, it reported that compound A can protect cerebral ischemia/reperfusion (I/R) injury within 24 hours by inhibition acute inflammation and apoptosis (Xu et al., Planta Medica, 2008, Vol. 74(8), pp. 816-821).

Reactive astrogliosis is a common pathological process in late phase of cerebral I/R injury, which contributes to further neuronal damages. It is also seen in neuronal degenerative disease such as Alzheimer's disease in the present invention, compound A given consecutively for 7 days in cerebral I/R injured rats. Results showed that compound A, exhibited protective effect against later phase cerebral I/R injury after 7 days as indicated by reduction of the infarct volume, improvement of the neurological behavior and cellular morphology, enhancement of the neuronal survival and reactive astrogliosis. The therapeutic effects of either single or consecutive 7 treatments with compound A were Analyzed and compared at 7 days after I/R injury. Consecutive 7 treatments with compound A significantly improved the I/R injury comparing to single treatment. Accumulation of activated astrocytes was found at 7 days after I/R injury, which was significantly inhibited by consecutive treatments with compound A.

The protective mechanism of compound A against the delayed phases of I/R injury is different that the acute phase in prior art. The later phase benefit mainly involves inhibition of reactive astrogliosis. As noted above, compound A the can increase cGMP by inhibition of PDE. It is known that cGMP can inhibit astrogliosis induced by cerebral injury, which may be mechanism of action of compound A.

Compound B of formula (I) has similar effects as compound A but often with less potency.

Compounds of formula (I) including compound A and B can also be used in treatment of other diseases involved in fibrosis or over production of collagen such as to reduce scar tissue formation in skin wound healing, corner recovery, retina injury, lung fibrosis, emphysema and liver cirrhosis.

Compounds of formula (I) including compound A and B can form pharmaceutical acceptable salts with other material such as basic metals (e.g. sodium) and halogen. They can be combined with pharmaceutical carriers to formulate pharmaceutical compositions. Compounds of formula (I) and their pharmaceutical compositions can be administered by oral, intravenous, inhalation, or other routes, and administered by catheter intervention into veins and arteries.

In other embodiment, compound A sodium was dissolved in sterile saline solution in a container connected with aerosolizer powered by compressed air (PARI nebulizer device). The aerosol droplets were evaluated using an impactor (NGI) in vitro to sure that the size of aerosol particles meet pharmaceutical standards (FDA or EU) in order of better lung deposition. Guinea pigs were anesthetized and the aerosol of compound A nebulization solution were delivery and inhaled into the lungs via a trachea tube. The therapeutically effects of compound A on lung function, fibrosis or inflammation of lungs were examined before and after scarification of animals. In prior art, compound A has never been used as inhaled medicine.

Further, this invention disclosed a medical suitable Intravenous injection formulation of compound A sodium, which is a liquid Formulation of compound A sodium using Co-solvent technology. Intravenous (i.v.) administration exerts quick therapeutic effects. However, i.v. administration of terpene such as compound A is highly limited by their low water solubility due to their chemical structures containing a hydrophobic hydrocarbon skeleton. A liquid pharmaceutical composition of compound A with sufficient stability and acceptable safety for i.v. administration has not been reported in prior art. For medical purpose, a pharmaceutical injectable formulation subjected to stringent test based on its toxicity, compatibility with solvent and stability under harsh conditions as well as pharmacokinetics in according to regulations of drug authorities. A medical suitable injectable pharmaceutical formulation of compound A has never been developed in prior arts. In this invention, for the first time, invented a pharmaceutical formulation of compound A which has physiological acceptable pH, compatibility with dilutes, sufficient physic-chemical stabilities and proved biological safety profile.

There are varieties of solubilization methods for hydrophobic compounds including use of surfactants, incorporation of hydrophobic compounds in nanoparticulate systems (e.g. liposomes, micelles and microemulsions) and cyclodextrin. However, surfactants are very limited for i.v. administration due to their toxicity and nanoparticulate systems are known to be challenging for clinical applications.

In the present invention, a liquid formulation of compound A sodium for i.v. administration was developed by tuning pH value and using low amounts of organic solvents that are well-accepted for pharmaceutical industry and clinics.

Only organic solvent which are already approved by FDA for i.v. administration were used for increasing solubility of compound A. After extensive screening of several solvents, the invention disclosed an optimized solvent system for compound A, which composed of saline at pH 10.0, 25% of ethanol and 20% of propylene glycol (2%, w/w) (compound A sodium). Compound A sodium was well solubilized in the invented formulation at maximum concentration of 20 mg or 50 mg/mL which minimized the use of solutes and avoid adverse effects, and this optimized formulation of the invention was physicochemical stable for at least 90 or 30 days without either crystallization or degradation during acceleration test with high humidity and high temperature conditions. Sterilization of autoclaving was conducted to ensure the safety of the formulation for i.v. injection, and compound A sodium was compatible and stable with the sterilization process.

This injectable formulation was shown to be stable during storage at low and high temperatures. Only negligible amounts of impurities were generated during the acceleration and long-term studies with harsh conditions involved, and both impurities and contents were in the acceptable range according to FDA guidelines. The hemolytic effect and cyto-compatibility of compound A were examined in this invention. The formulation did not induce either hemolytic effects up to 9.1% (v/v) for 3 hours or significant cytotoxicity up to 50 μg/mL in H2C9 cells. In vivo study that no significant acute toxicities were observed in rats received excessive amount of the formulation. These tests indicate the injectable formulation of this invention has a pharmaceutically acceptable safety.

The pharmaceutically acceptable salts of compound of formula according to the invention include those formed with conventional pharmaceutically acceptable inorganic or organic acids for example: sodium, hydrochloride, hydrobromide, sulphate, hydrogen sulphate, dihydrogen phosphate, methanesulfonate, bromide, methyl sulphate, acetate, oxalate, maleate, fumarate, succinate, 2-naphthalene-sulphonate, glyconate, gluconate, citrate, tartaric, lactic, pyruvic isethionate, benzenesulphonate or p-toluenesulfonate.

Above is a general description of the invention. The methods and technologies according to the invention are better illustrated by the following examples, so that they can be performed by a skilled person in art.

The methodologies and embodiments of this invention are provided in detail in the following examples.

### EXAMPLES

To further illustrated the technologies used to achieve the objects of the invention, a detailed methods, techniques, procedures, and special features regarding in determining and identifying the pharmaceutical and therapeutic usefulness of kaurane compounds in this invention are described bellow.

Examples provide experimental methods and results which are utilized for supporting the invention, and for validating the animal models used in the invention. Proper control and statistic testing are used in all the experiments in this invention. The following examples are provided to illustrate, not limit, the invention. The examples illustrate the methods and techniques utilized to screen and to determine the therapeutic use of some kaurane compounds in the compounds of formula (I). The therapeutic use of other compounds of formula (I) can also be determined in the same way.

### Experiment Materials

Animal: Adult male Wistar rats, weighing 200 g±20 g, 9 weeks old, both sexes. Each rat was housed in an individual cage under standard conditions, constant temperature and humidity, and a strict dark-light regiment, and received standard laboratory diet ad libitum. Chemical: Compound A (ent-17-norkaurane-16-oxo-18-oic acid, molecular formula, C₂₀H₄₀O₃, Molecular weight: 318.5) is produced from stevioside through acidic hydrolysis, crystallization and purification. The sodium salt of compound A can be formed by adding NaOH or other sodium containing base. The structure of compound A are confirmed by inferred analysis and NMR, which are consistence with previously published data. The sodium salt of compound A formed by the purity of compound A is greater than 99% determined by high performance liquid chromatograph. Compound B (ent-13-hydroxykaur-16-en-18-oic acid) is produced from stevioside through a series processes including oxidation, hydrolysis, acidification, extraction, purification and crystallization. The structure of compound B is confirmed by inferred analysis and NMR, which are consistence with previously published data. (Mosettig E. et al., 1963). The purity of compound B is greater than 99% as determined by high performance liquid chromatograph. Administration of testing compounds: intravenous or intraperitoneal injection or oral. Dosage: compound A: 0.5 mg/kg to 10 mg/kg (or its sodium salt); compound B: 2 mg/kg to 20 mg/kg.

### Experimental Methods

Establishment of Cardiac Hypertrophy (TAC) Animal Model and Experimental Protocol

TAC between the innominate artery and the left carotid artery was conducted to induce pressure overload for 3 weeks (3-week TAC) or 9 weeks (9-week TAC). Sham control animals underwent the same operation, but without aortic constriction. All surgical procedures were performed with animals anesthetized with 3% pentobarbital sodium injected intraperitoneally (i.p. 40 mg/kg). During the surgery period, rats were intubated and ventilated with a rodent ventilator (Harvard Apparatus, Holliston, Mass., USA).

Both 3-week and 9-week TAC-exposed rats were randomly divided into five groups (n=8-10 rats) including a TAC vehicle control, COMPOUND A, low (TAC+COMPOUND A (L), 1 mg/kg/d), middle (TAC+COMPOUND A (M), 2 mg/kg/d), high (TAC+COMPOUND A (H), 8 mg/kg/d) dose group respectively, and sildenafil (TAC+Sil, 70 mg/kg/d) as a positive control group. Sham controls were all treated with vehicle. Acute and chronic mortality from the TAC procedure was <5%. The treat group was intra-gastric administrated with sodium salt of compound A which was solved in a mixture of saline and organic solvent of the same volume (1:1, 0.5 ml) and sildenafil which was solved in distilled water. All drugs and vehicle treatment were given twice a day after surgery for three days as designed. The animals were examined at 3 weeks and 9 weeks after surgery accordingly. At the end of the observation periods and after hemodynamic measurement in vivo, all animals were sacrificed and hearts were explanted for further Analyses.

Measurements of Cardio-Dynamic Parameters

Heart hemodynamic Analysis was conducted by pressure-volume (PV) catheter. Rats were anesthetized and placed on a warming pad (37° C.). Underwent tracheostomy, rats were then ventilated by using a positive pressure with a tidal volume of 4-6 ml/200 g at 70 breaths/min using room air. The right internal carotid was identified and ligated cranially. A four-electrode pressure-volume catheter (model SPR-838, Millar Instruments Inc.) was advanced into the right carotid artery without open-chest and then advanced into the left ventricle until stable PV loops were obtained. After stabilization of the signal for 10-15 min, baseline PV loops were recorded at a steady state. The abdomen was then opened to identify the inferior vena cava and portal vein. Preloads during the vena cava occlusions were varied by compression of the inferior vena cava with a cotton tip applicator. During the data collection, the small animal respirator was shut down for 5 s to avoid artifact from lung motion. After data were recorded under steady-state conditions and during preload reduction, parallel conductance values were obtained by the injection of 40 μl hypertonic saline (30%) into the right jugular vein. Calibration from relative volume unit conductance signal to absolute volumes was undertaken by using a previously described method. During the measurement of left ventricular function in vivo, changes in peripheral resistance in each animal were examined. Through a longitudinal inguinal incision, a polyethylene arterial catheter (PE10) connected to a pressure transducer was inserted into the distal abdominal aorta via the femoral artery retrograde. Data were recorded on separate channels of the PowerLab system. The catheter was filled with heparin saline (100 U/ml) to prevent blood coagulation.

Histology Study

Tissue sections of rat hearts were fixed in 10% neutral-buffered formalin, embedded in paraffin, cut into 3 mm serial sections, and then stained with haematoxylin and eosin (H&E), picrosirius red or phalloidin. Nikon system and Zeiss confocal microscope were used to capture digital images. Stained with H&E was to evaluate cell size, stained with picrosirius red (sigma CA) was to test fibrosis using standard procedure and the amount of F-actin was stained with phalloidin. We determined cross sectional cell area and interstitial collagen fraction using computer-assisted image analysis (Image-Pro Plus software), with the observer blinded as to tissue source. At least four or five different hearts were quantified for analysis.

Isolation and Culture of Cardiac Fibroblasts

Neonatal rat cardiac fibroblasts were isolated from 1-2-day-old Sprague-Dawley rats as described previously. Briefly, hearts from newborn 1-2-day-old Sprague-Dawley rats were minced on ice, and cells were isolated by trypsin incubation at 37° C. Non-cardiomyocytes were separated from the cardiomyocytes by differential pre-plating, and then cardiomyocytes were removed with fibroblasts seeded in culture dishes. The cells were passaged after 3 days, using a 0.05% trypsin solution. Cells were cultured in DEME/F12 medium with 5% fetal calf serum, and maintained at 37° C., 5% CO₂ condition.

Cell Proliferation

Viability of cardiac fibroblast in culture was assessed using the 3-(4,5-dimethylthiazol-2-yl)-2,5-diphenyltetrazolium bromide (MTT) method. The assay measures the ability of an active mitochondrial enzyme to reduce the MTT substrate (yellow to blue) in live cells. Isolated primary cardiac fibroblasts were plated in serum-free conditions on 96-well plates. After 24 h of culture, 0.5 mg/ml MTT substrate added and cells were incubated for additional 4 h, and then solubilized with DMSO 10 min at room temperature. Absorbance was measured at 460 nm.

Statistical Analysis

All data were presented as mean±s.e.m. Differences between multiple groups were compared by analysis of variance (one way ANOVA) followed by a Fisher test. All P values were 2-sided. Values of P less than 0.05 were considered statistically significant.

### Example 1

This example illustrates the effects of COMPOUND A on reduction of TAC-induced cardiac hypertrophy and cardiomyocyte dilation.

Adult Wistar rats were subjected to TAC for 3 weeks and treated with vehicle, COMPOUND A or sildenafil, respectively. A significant increase in heart to body weight ratio (HW/BW), an index of cardiac hypertrophy, was observed in the 3-week TAC group (increase 34.6%, P<0.001), which was accompanied by increased cardiac cross-sectional area (increase 81.6%, P<0.001). The cardiac and myocyte hypertrophy were ameliorated by compound A or sildenafil in the 3-week TAC groups (Table 1). The increment of cardiomyocyte cross-sectional area was reduced to 15.1 (1 mg/kg) and 4.1% (2 mg/kg) by compound A and 16.3% (70 mg/kg) by sildenafil respectively. Compound A is more potent and effective that sildenafil.

TABLE 1
Effects of compound A on Heart weight to body weight in TAC rats (n = 8)

| | Sham | TAC | TAC + comp. A(L) | TAC + Sil |
|---|---|---|---|---|
| 3 wk | | | | |
| HW(g) | 0.68 ± 0.03 | 0.91 ± 0.06*** | 0.72 ± 0.03^{##} | 0.76 ± 0.04^{#} |
| BW(g) | 272.6 ± 10.82 | 270.75 ± 8.41 | 250.5 ± 5.17 | 264.5 ± 7.9 |
| HW/BW(mg/g) | 2.497 ± 0.101 | 3.361 ± 0.155*** | 2.862 ± 0.099^{##} | 2.86 ± 0.117^{##} |

### Example 2

This example illustrates the effects of compound A inhibit actin remodeling and fibrosis formation.

Some transcription factors important for hypertrophy influence actin dynamics, which is regulated by free G-actin and polymeric F-actin. A higher F-to-G actin content is an important result of the activation of hypertrophy pathways. The level of myocardia F-actin was measured by FITC-phalloidin staining. The representative immunofluorescence image of TAC showed an intensified green staining of F actin after 9 weeks, which was returned to control conditions by treatment with compound A (8 mg/kg/d) or sildenafil (70 mg/kg/d). TAC increased the level of F actin, thus lead to actin dynamics. Both compound A and sildenafil can reduced the expression of F actin and maintain F/G actin balance.

To determine whether compound A attenuates TAC-induced cardiac fibrosis, heart tissues were stained with picrosirius red to detect interstitial collagen distribution in left ventricular. In both 3-week and 9-week TAC groups, TAC induced significant interstitial fibrosis (P<0.05). The collagen content increased 5.7 fold and 7.5 fold in 3-week and 9-week TAC control groups, respectively, compared to sham control group. Compound A (8 mg/kg/d) treatment resulted in 58.2% and 80.8% reductions in interstitial fibrosis in 3-week and 9-week TAC groups, respectively. Sildenafil exhibited less inhibition effect on cardiac fibrosis compared to compound A.

### Example 3

This example illustrates the effects of Compound A on production of cGMP.

Measurement of cGMP

cGMP levels in the neonatal rat fibroblasts after treated with vehicle or compound A or sildenafil were measured with an ELISA kit following the manufacture's instruction. Quiescent cells were cultured with different doses of compound A (1M, 10M) or sildenafil (100M) for 3 h. After treatment, the cells were lysed with 0.1N HCl, and performed cGMP ELISA assay. The results are listed in table below.

TABLE 1
Stimulated Production of cGMP by compound A
and Sildenafil (percent of control)

| | |
|---|---|
| Control | 1.00 ± 0.00 |
| Compound A-1Na 1 um | 1.57 ± 0.43 |
| Compound A-1Na 10 um | 2.07 ± 0.54 |
| sildenafil 100 um | 1.41 ± 0.27 |

### Example 4

This example illustrates compound A stables the impaired cardiac autonomic balance by TAC by suppressing the sympathetic activities.

Electrocardiograph Monitoring

Three or nine weeks after TAC operation, rats were anesthetized with pentobarbital sodium (i.p. 40 mg/kg). The electrocardiogram (ECG) was measured using the II Einthoven lead. Three stainless steel 22G needle electrodes were localized in the insertion of the right (G1) and left (GND) front legs, and in the left (G2) rear leg. Accordingly, 10 min of ECG recordings were digitally acquired at 2 kHz prior to any maneuver. Heart rate variability-Spectral Analysis was performed using fast Fourier transformation. Oscillatory components were separated into very low frequency (VLF; <0.04 Hz), low frequency (LF; 0.04-0.6 Hz), or high frequency (HF; 0.6-2.5 Hz) bands. HRV components were expressed in normalized units (n.u.) as a percentage of total power minus the VLF component. Efferent vagal parasympathetic activity is a major contributor to the HF component and both sympathetic and vagal influences contribute to the LF component; thus the ratio of LF to HF is commonly utilized as a measure of sympathovagal balance.

Parameters of the heart rate variability (HRV) are indicators for cardiac autonomic balance. The power spectrum analysis of RR variability shows that rats exposed to TAC for 9 weeks displayed marked changes in the distribution of the relative spectral components of HRV. The LF/HF ratio was marked higher compared to sham controls, while LF/HF ratio was reversed to normal by compound A treatment (P<0.01). Sildenafil treatment did not reduce LF/HF ratio. This invention disclosed a novel used of compound A for restore cardiac autonomic balance by suppress elevated sympathetic activity, which sildenafil had no such effect.

### Example 5

This example illustrates compound A improves ECG alterations induced by TAC.

We further studied the effects of compound A on electrophysiological alterations in the hypertrophic heart. TAC-exposed rats at 9 weeks had a longer QRS duration and higher R amplitude (P<0.05). After compound A or sildenafil treatments, QRS duration and R amplitude trended normal. Significant increase in QT dispersion (P<0.01) and QTc dispersion (P<0.01) induced by TAC surgery after 9 weeks, which indicated a high risk of cardiac arrhythmias. Compound A treatment reversed such effects whereas sildenafil treatment did not show similar protective effect.

### Example 6

This example illustrates compound A improves cardiac function in cardiomyopathy and prevents cardiac remodeling, fibrosis and inflammation from diabetes injury.

Diabetic cardiomyopathy (DCM) Diabetic induced injury to the myocardium. DCM, induced by streptozotocin (STZ), along with the associated changes occurring in inflammation, oxidative stress and fibrosis markers. Wistar rats were randomly divided into four groups: group A (Normal control), group B (Diabetes), group C (DM/STVNa) and group D (DM/TMZ, trimetazidine treatment). After 12-16 weeks, left ventricular function was measured by the pressure-volume system. Cardiac tissues were prepared for histological study by hematoxlyin and eosin, Sirius red staining as well as for assays of oxidative stress. Oxidative stress, inflammation, and fibrosis markers were evaluated by molecular biological techniques. All data were measured morphometrically and statistically analyzed. All treated groups showed a significantly increase blood glucose and decrease in insulin levers comparing to control. The diabetes group showed cardiomyocytes hypertrophy, inflammations, interstitial fibrosis, significant increases in the collagen volume fraction, TGF 3 and oxidative stress in cardiac tissues, as well as decreased superoxide dismutase 2 (SOD-2) expression and activity compared with normal groups. Compound A as well as TMZ treatment significant inhibited cardiac hypertrophy, the relative heart weight and antioxidant activities in group C and D were similar to the control. However, there were no significant changes in blood glucose level and insulin levels in groups B and D in comparing to Diabetes group (B). The cardiac function was significantly improved in groups B and D comparing to group B.

This invention disclosed that compound A can prevent the cardiac injury, cardiac remodeling and fibrosis induced by diabetes and can improve the cardiac function of cardiomyopathy in debates and these effects is not related to changes in either glucose or insulin.

### Example 7

This example illustrates the effects of compound A in treatment of pulmonary hypertension.

Pressure overloaded-induced pulmonary hypertension was induced by transverse aortic constriction in male wistar rats (200±20 g body weight). Sham operated rats served as controls. Compound A treatment (2 or 4 mg/kg body weight daily intra-gastric lavage) was initiated 3 days after aortic constriction and continued for 9 weeks. After 9 weeks, animals were sacrificed and lungs were fixed, paraffin-embedded, sectioned, and stained with hematoxylin and eosin. Blinded stereological Analysis of mean wall thickness and vascular muscularization of lung arterial vessels was performed. Collagen I was verified by immune-fluorescent staining and visualized in confocal.

### Results

1) Lung Vascular Remodeling

Considerable lung vascular remodeling was evident in pulmonary hypertension rats as medial wall thickening in small (inner diameter <100 um), medium pulmonary arteries (diameter <100 um). Compound A prevented vascular remodeling in small and medium arteries.

TABLE 1
The comparison of mean vascular wall thickness
of pulmonary arterioles with an
inner diameter <100 um ( x̅ ± SD, n = 3)

| | Mean vascular |
|---|---|
| Group | wall thickness |
| Sham | 24.39 ± 7.87 |
| TAC | 30.49 ± 8.51 ** |
| 1 mg/kg COMPOUND A | 22.96 ± 7.83^{##} |
| 4 mg/kg COMPOUND A | 17.60 ± 6.28^{##&&} |
| Sildenafil | 24.10 ± 7.48^{##} |

Note:
** p <0.01 compared with sham group.
^{##}p <0.01 compared with TAC group.
^{&&}p <0.01 compared with sildenafil group.

TABLE 2
The comparison of mean vascular wall thickness
of pulmonary arterioles with an
nner diameter >100 um ( x̅ ± SD, n = 3)

| | Mean vascular |
|---|---|
| Group | wall thickness |
| Sham | 16.53 ± 7.45 |
| TAC | 24.75 ± 8.40* |
| 1 mg/kg compound A | 16.60 ± 6.00^{##} |
| 4 mg/kg compound A | 10.67 ± 5.01^{##} |
| Sildenafil | 14.88 ± 6.83^{##} |

Note:
*p < 0.05 compared with sham group.
^{##}p < 0.01 compared with TAC group.
^{&&}p < 0.01 compared with sildenafil group.

2) Vascular Muscularization

According to the diameter of pulmonary vascular, there are there different extents of vascular muscularization, non-muscularization, partially muscularization and fully muscularization. After treatment of compound A, the number of non-muscularization vessels was increased, indicating the amelioration of pulmonary hypertension. Compound A is significantly more potent and effective comparing the sildenafil group.

TABLE 3
The percentage of different extents of vascular muscularization in the five
groups of rats (x̅ ± SD, n = 3)

| | Non- | Partially | Fully |
|---|---|---|---|
| Groups | muscularization | muscularization | muscularization |
| Sham | 72.38 ± 10.91 | 18.47 ± 5.822 | 9.147 ± 7.620 |
| TAC | 30.78 ± 15.96* | 27.12 ± 8.217 | 42.08 ± 10.72* |
| Compound A | 66.78 ± 5.876^{#} | 23.00 ± 3.938 | 10.20 ± 9.787^{#} |
| 1 mg/kg + TAC | | | |
| Compound A | 81.10 ± 16.60^{#} | 17.04 ± 18.98 | 1.851 ± 3.207^{#&} |
| 4 mg/kg + TAC | | | |
| Sildenafil | 57.57 ± 7.182^{#} | 18.61 ± 15.09 | 23.80 ± 8.247^{#} |
| 70 mg/kg + TAC | | | |

Note:
*p < 0.05 compared with sham group.
^{#}p < 0.05 compared with TAC group.
^{&}p < 0.05 compared with sildenafil group.

3) Immunofluorescence Staining of Collagen I

Collagen I expression in the lungs is assessed Fluorescence imaging of collagen I identified a marked increase in the lung tissue of TAC group, compared with that of sham group. Compound A treatment reduced the production of collagen I.

TABLE 4
The fluorescent intensity of collagen I in different groups

| Groups | Collagen I |
|---|---|
| Sham | 7.518 |
| TAC | 15.88 |
| Compound A | 10.75 |
| (1 mg/kg) + TAC | |
| Sildenafil | 7.591 |
| (70 mg/kg) + TAC | |

1. A method of treatment or prevent of cardiomyopathy or cardiac hypertrophy or pulmonary hypertension or fibrosis remodeling of normal tissues and other related diseases, via a mechanisms involve TGF-β, microRNA and novel phosphodiesterase modulation or their combine, and comprising use of kaurane compounds or their pharmaceutical acceptable salts in manufacture of specific pharmaceutical standard solid or liquid composition for administering to a patient in need.

2. The methods of claim 1, wherein the said cardiac hypertrophy were characterized by increasing in cardiac mass, size of myocardium, and sympathetic activities, and over-production of myofibroblasts, collagen and B-type natriuretic peptide.

3. The methods of claim 1, wherein the said cardiac hypertrophy is the result of hypertension, ischemia, cardiomyopathy, diabetes and other cardiac diseases.

4. The methods of claim 1, wherein the said pulmonary hypertension is due to pulmonary arteriopathy including intima proliferation, median hypertrophy and vascular muscularization, increase in fibrosis, extra cellular matrix and collagen production.

5. The methods of claim 1, wherein the said pulmonary hypertension is the result from hypertension, hypoxia or lung diseases.

6. The methods of claim 1, wherein the said fibrosis remodeling were increase in fibroblast cells and over-expression of extra cellular matrix or collagen.

7. The methods of claim 1, wherein the said fibrosis remodeling includes liver or kidney cirrhosis or retina fibrosis and wound healing from surgery or trauma in skin, intestine, cerebral and other organs, vascular or coronary angioplasty.

8. The methods of claim 1, wherein the said method for treatment and prevent disease involves a stimulation of cGMP production in the disease been treated.

9. The methods of claim 1, wherein the said method for treatment and prevention cardiac hypertrophy involves improve cardiac function without further increasing oxygen consumption and worsening existing ECG.

10. The methods of claim 1, wherein the said method for treatment and prevention involves a stimulation of cGMP production and/or modulation of microRNA21 in the disease been treated.

11. The methods of claim 1, wherein the said method for treatment and prevention involves using a novel phosphodiesterase inhibitor which characterized by increasing cGMP production and reducing reactive oxygen species level in cells in the disease been treated.

12. The methods of claim 1, wherein the said method for treatment and prevention involves using a novel phosphodiesterase modulator which characterized by changing in 2′3′ cGMP, 3′5′cGMP, 2′3′cAMP and 3′5′cAMP production or their ratios in cells in the disease been treated.

13. The methods of claim 1, wherein the other related disease is erection dysfunction.

14. The methods of claim 1, wherein the other related disease are and neurodegenerative diseases and metabolic disorders.

15. The methods of claim 1, wherein the other related disease is abnormal ECG with long QT segment or increased QT variation as results of elongated action potential, depolarization, and suppressed Herg current.

16. The methods of claim 1, wherein the other related disease is enhanced cardiac sympathetic activity.

17. The methods of claim 1, wherein the other related disease involves activation of astrogliosis including neurodegenerative disease, senile dementia; Alzheimer's disease and late phase of cerebral injury.

18. The methods of claim 1, wherein, said compounds is compound A presented in the structure formula (I)

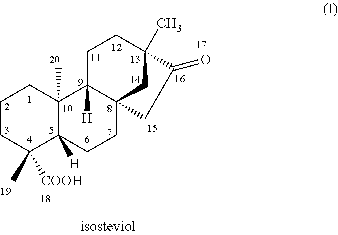

19. The method of claim 1, wherein, said compounds is compound B presented in the structure formula (II)

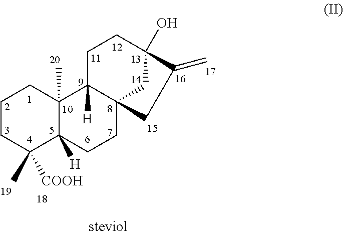

20. The method of claim 1, wherein the said solid pharmaceutical compositions are selected from the group consisting of tablets, capsules, granules, suppository, ointment, time-released dosage forms for oral, parenteral or implant use.

21. The method of claim 1, wherein the said pharmaceutical compositions are selected from the group consisting of inhalation nebulizer, MDI or dry powders for pulmonary or spray for nasal delivery.

22. The methods of claim 1, wherein the said specific pharmaceutical standard compositions is pharmaceutical standard aqueous liquid injectable aqueous liquid formulation or suitable dosage forms delivered via catheter intervention consisting of saline and organic solvent or solvent mixture as solute or solubilizing agents.

23. The methods of claim 22, wherein the said solvents are ethanol, 1,2-propylene glycol, glycerol, polyethylene glycol or other pharmaceutical suitable organic solvents and the volume of each of the solvent in a mixture is from 5% to 90%.

24. The methods of claim 22, wherein the said solubilizes include alcohols (chlorobutanol), dioxolanes, ethers, glycerol, amides (dimethylacetamide), esters (ethylis oleas), plant oils (soybean oil), sulfoxides (dimethyl sulfoxide), and polymeric compound (Kolliphor RH40) and other pharmaceutical suitable solubilize agents.

25. The methods of claim 22, wherein the said specific pharmaceutical standard pharmaceutical injectable aqueous liquid formulation meets the stability and biological safety requirements of pharmacopeias published by drug authorities in US, EU, Japan and China.
